(19) United States
(12) Patent Application Publication
(10) Pub. No.: US 2008/0207986 A1
(43) Pub. Date: Aug. 28, 2008

(54) Heart assist device

(76) Inventor: Daniel S. J. Choy, New York, NY (US)

Correspondence Address: STEVEN L. NICHOLS;RADER, FISHMAN & GRAUER PLLC, 10653 S. RIVER FRONT PARKWAY, SUITE 150, SOUTH JORDAN, UT, 84095, US

(21) Appl. No.: 11/710,698

(22) Filed: Feb. 26, 2007

(51) Int. Cl. A61N 1/362

(52) U.S. Cl. 600/16

(57) ABSTRACT

A heart assist device and method of making the same includes a catheter and a balloon attached to the catheter. The heart assist device is used with a system for inflating and deflating the balloon in sequence to systole and diastole of a patient's heart. In some examples, the catheter has a curved portion with a stiffening agent disposed therein. In some examples, a tip of the catheter extends into an interior of the balloon.

### BACKGROUND

As a heart pumps blood, it both expands to draw in blood and contracts to expel blood. The act of drawing blood into the heart is referred to as diastole. The act of expelling blood from the heart is referred to as systole.

In certain pathological conditions, the heart, and principally the left ventricle, cannot contract fully during systole. Consequently, there is incomplete emptying of the blood from the ventricle. The amount of blood remaining in the ventricle at the end of systole represents unused pumping capacity and may be referred to as “dead volume” or “dead space.”

The inability to fully contract during systole typically results from damage to the left ventricular muscle. Such muscular damage may arise from a variety of causes, including, chemical, physical, bacterial or viral. As noted, any such damage to the left ventricular muscle typically leads to a decrease of contractility and therefore a decrease of blood ejection function during systole.

The inability of the left ventricle muscle to fully contract during systole frequently leads to congestive heart failure. Such heart failure may be correctable to varying degrees by pharmacological or mechanical intervention. However, in intractable left ventricle failure, when it is not possible to increase the stroke volume, the dead volume or space continues to remain at the end of the systole.

In such cases, the prior art teaches a ventricular assist device that can be inserted into the left ventricle or other portions of the heart to assist the ventricle or other muscle to draw or expel blood, thereby eliminating the lost pumping capacity or “dead volume.” Such heart assist devices are disclosed in U.S. Pat. No. 4,685,446, entitled “Method for Using a Ventricular Assist Device” to Choy and U.S. Pat. No. 4,902,273, entitled “Heart Assist Device” to Choy et al., both of which are incorporated herein by reference in their respective entireties.

### SUMMARY

A heart assist device includes a catheter and a balloon attached to the catheter. The heart assist device is used with a system for inflating and deflating the balloon in response to systole and diastole of a patient's heart. In some examples, the catheter has a curved portion with a stiffening agent disposed therein. In some examples, a tip of the catheter extends into an interior of the balloon.

A method of making a heart assist device includes providing a catheter, wherein the catheter has a curved portion with a stiffening agent disposed therein; and attaching a balloon to the catheter that can be inflated with a fluid or gas provided through the catheter.

### BRIEF DESCRIPTION OF THE DRAWINGS

The accompanying drawings illustrate various embodiments of the principles described herein and are a part of the specification. The illustrated embodiments are merely examples and do not limit the scope of the claims.

FIG. 1 is a front perspective view of an improved ventricular assist device;

FIG. 2 is a view of the heart showing the device in a temporary installation passing through the Aortic valve in its inflated state in solid line and in its deflated state in dotted line.

FIG. 3 is a view similar to FIG. 2, but primarily of the left ventricle showing the device in a permanent installation passing through the Mitral valve in its inflated state in solid line and in its deflated state in dotted line.

FIG. 4 is a view showing the balloon in the collapsed configuration folded back along the catheter for insertion;

FIG. 5 is a view similar to FIG. 4 with the balloon partially inflated, to the operating and inflated position;

FIG. 6 is a view similar to FIG. 5 with the balloon further inflated;

FIG. 7 shows the balloon at the distal end of the catheter in its operating, fully inflated state;

FIG. 8 is a schematic view of a structure permanently implanted in subcutaneous fat but with external electrical contacts;

FIG. 9 is a schematic view of another structure completely permanently implanted in the subcutaneous fat;

FIGS. 10, 11, 12, 13 and 14 are views of the balloon packaged in another manner to facilitate insertion via an artery or through the left atrium, from the deflated state, and with inflation, gradually extending itself beyond the end of the catheter;

FIG. 15 is a graph illustrating Aortic flow;

FIG. 16 is graph illustrating intraventricular pressure;

FIG. 17 is a view similar to FIG. 2 but shows a ventricular assist device inserted into the left ventricle through the apex of the left ventricle; and

FIG. 18 is a flow chart illustrating a method of using a heart assist device according to principles described herein.

FIG. 19 is a schematic view of a ventricular assist device according to the principles of the present specification.

FIG. 20 is a schematic view of another heart assist device according to principles described herein.

FIG. 21 is a schematic view of still another heart assist device installed in a heart according to principles described herein.

Throughout the drawings, identical reference numbers designate similar, but not necessarily identical, elements.

### DETAILED DESCRIPTION

The present disclosure describes to a method for using a ventricular assist device, and more specifically is directed to a device in which the expandable member is placed directly within the left ventricle of the heart to facilitate increased ejection of the blood during systole.

The following specification describes a method for using a ventricular assist device having a catheter with a proximal end and a distal end, a pump secured to the proximal end of the catheter, and an inflatable balloon secured to the distal end of the catheter. The balloon is inserted into the left ventricle of a patient's heart. The balloon is inflated during left ventricular systole, and then the balloon is deflated rapidly, just prior to the onset of the diastole. The inflating and deflating steps are repeated. Preferably, the inflating step starts at approximately the beginning of left ventricular systole and stops at approximately the end of left ventricular systole. The balloon may be inserted into the heart through the mitral valve, through the aortic valve, or through the apex of the left ventricle. The pump is advantageously implanted within the patient's body, e.g., within an envelope of skeletal muscle.

The ventricular assist device may include a shaped radioopaque balloon connected to the tip of an intra-arterial catheter with a single lumen. The proximal end of the catheter is connected to a gas pump that is capable of inflating and deflating the balloon in a range of 50 to 120 cycles per minute. The gas used is either carbon dioxide or helium. The pump mechanism is triggered by an electronic relay connected to an electrocardiograph, so that inflation and deflation are governed by specific time sequences in the EKG corresponding to electrical systole and diastole.

The balloon is selected to properly fit within the left ventricular chamber, and is made to inflate just as mechanical systole begins. The cessation of inflation corresponds to the end of mechanical systole. Active contraction of the balloon begins just prior to the onset of mechanical diastole. The negative pressure thus generated increases the pressure gradient between the left atrium and left ventricle, thus augmenting diastolic filling. This sequence of events enables the balloon to expand meeting the incoming (contracting) walls of the ventricle, thus decreasing the dead space and augmenting stroke volume. Since the Mitral valve is closed, and the Aortic valve is open, all the blood ejected flows distally into the aorta in a physiologic manner.

While it is possible to operate the ventricular assist device by means of external manipulation as is done in prior art devices, it is preferred to have the device wholly implanted within the body of the user, requiring no external equipment for proper operation. This is possible by creating a muscle pump, for example, by using skeletal muscle with timed means to internally stimulate the muscle causing appropriate inflation and deflation of the balloon. Another modified embodiment uses a solenoid pump with contacts lying just on the outer surface of the skin, designed to be connected to an external power source. Thus, the unit can be either self-contained and has a “no tether” feature or a “no tube tether” feature.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “an embodiment,” “an example” or similar language means that a particular feature, structure, or characteristic described in connection with the embodiment or example is included in at least that one embodiment, but not necessarily in other embodiments. The various instances of the phrase “in one embodiment” or similar phrases in various places in the specification are not necessarily all referring to the same embodiment.

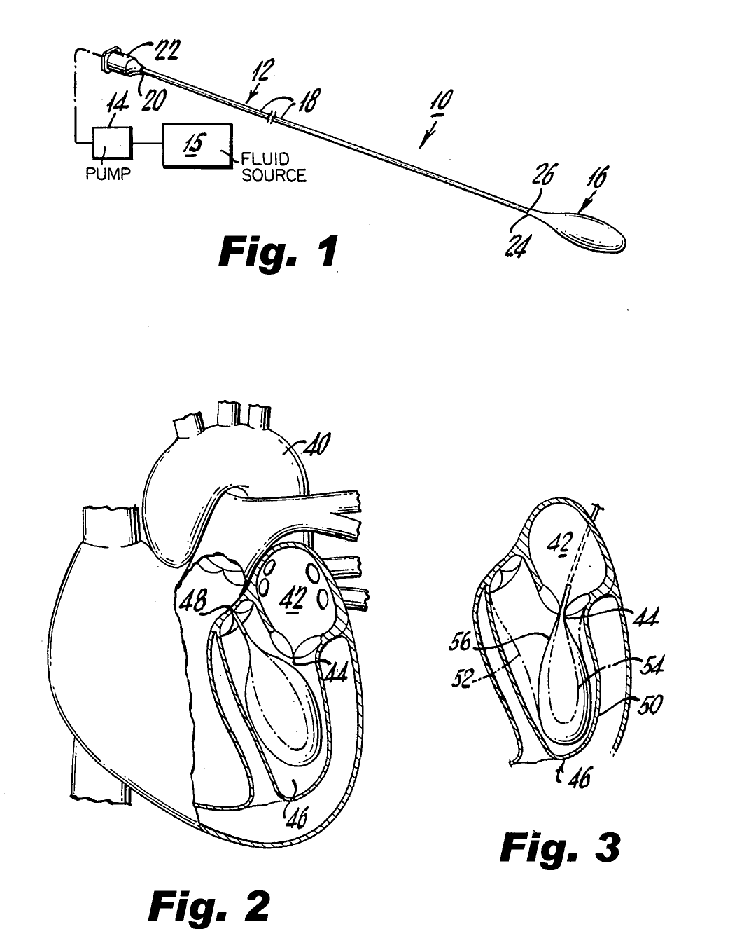

Referring to the drawings, and in particular to FIG. 1, there is shown an improved ventricular assist device 10 broadly comprising a catheter 12, a pump 14 (shown in schematic), a fluid source 15, and an inflatable balloon 16.

The catheter is generally made of plastic or a woven synthetic material and is a standard flexible hollow catheter defined by an outer surface 18, a proximal end 20 to which is secured an attachment member 22 for making a connection to the pump 14, and a distal end 24 having either a securement device or bonding means 26. The bonding means 26 is used to secure the balloon 16 to the distal end 24.

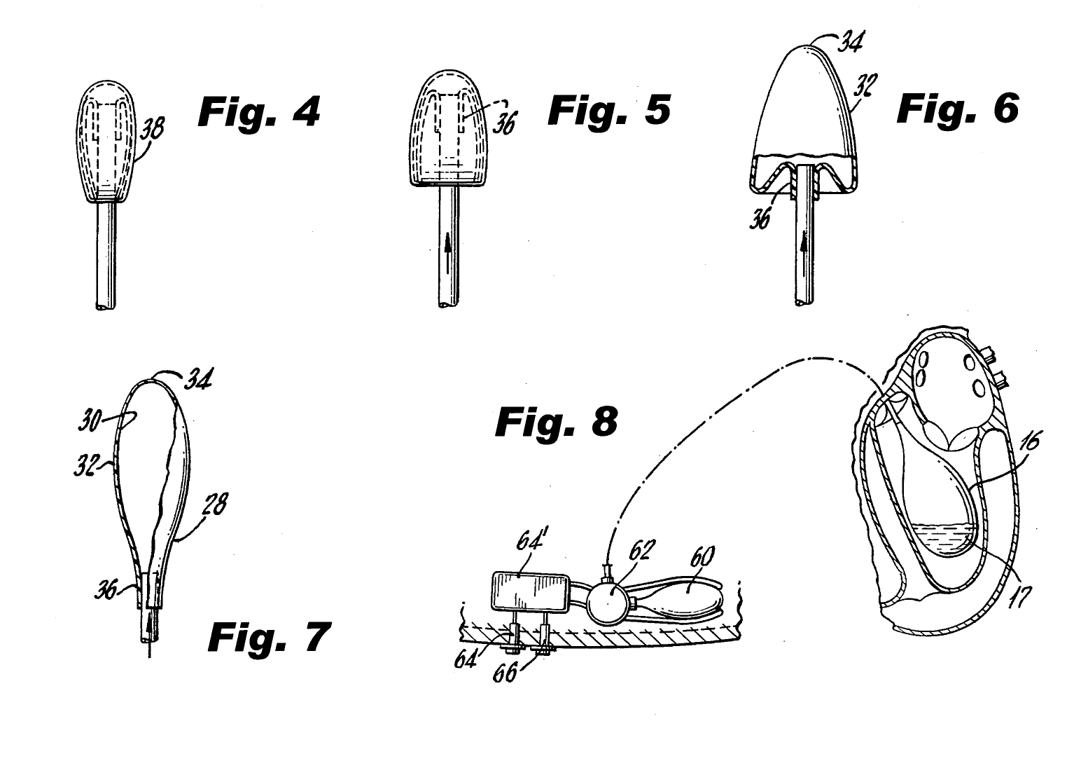

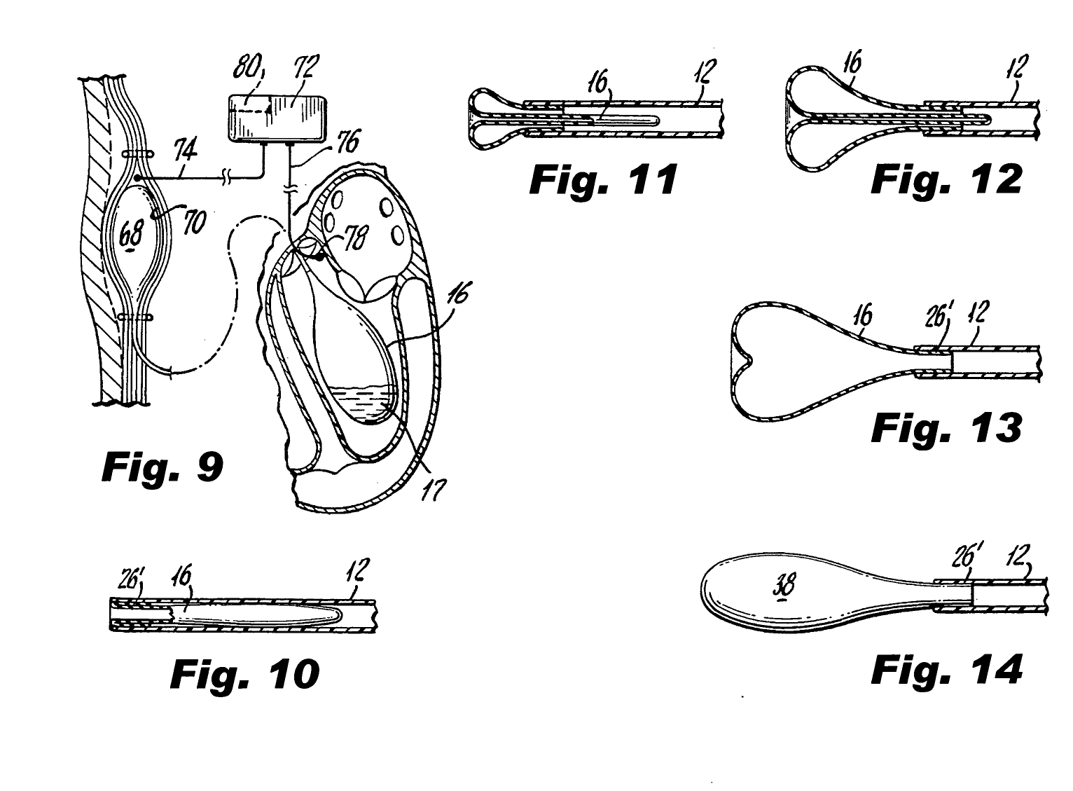

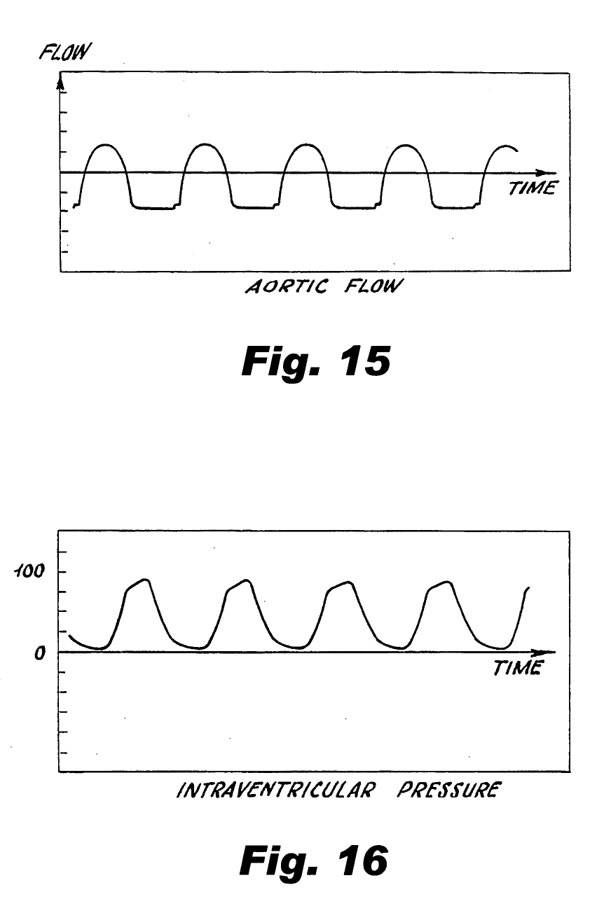

Turning more particularly to FIG. 7, the balloon includes a wall 28 defined by an inner surface 30 and an outer surface 32. For percutaneous insertion through a dilator: the balloon is folded so that it overlaps itself forming a crown 38 as in FIG. 4. Alternatively, it may be packaged inverted on itself inside the lumen of the catheter as in FIG. 10. Both configurations are to provide a minimal cross-sectional area to facilitate insertion. The balloon is securely attached to the distal end 24 of the catheter 18 by bonding 26, for example, as at 36 to provide an air-tight seal between the neck of the balloon and the catheter. FIGS. 5 and 6 illustrate the balloon during progressive stage of inflation.

The pump unit 14 is similar to existing pumps used to drive intra-aortic balloon assist devices and is activated at specific points in the cardiac cycle.

FIG. 2 shows a representation of the heart with the aorta 40 leading away from the opposite side of the left atrium 42. The left atrium ends at the Mitral Valve 44 which then leads into the left ventricle 46. The Aortic Valve 48 provides the exit from the left ventricle.

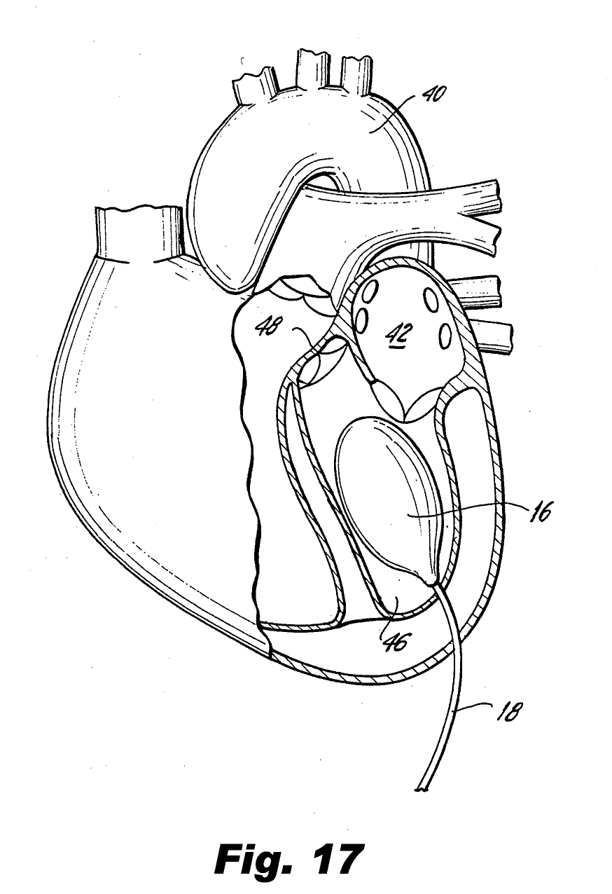

Turning to FIG. 3, there is shown a representation of the operation of the device being described. The outer solid line shows the maximum diastolic margin 50 of the inner ventricular wall and the maximum end systolic margin is shown in dotted line 52. The inflated balloon is shown in solid line 56 and the deflated balloon is shown in dotted line 54. The installation through the Mitral Valve as shown in this figure is a permanent installation as opposed to a temporary installation through the Aortic Valve illustrated in FIG. 2. A permanent installation may also be accomplished by inserting the balloon through the apex of the left ventricle, as shown in FIG. 17.

To prepare the heart assist device, the end-systolic and diastolic volumes and shapes of the left ventricle are determined by imaging techniques, such as two-dimensional echocardiography or isotope tomography.

For example, techniques for determining left ventricular volume are disclosed in an article entitled “Usefulness and Limitations of Radiographic Methods for Determining Left Ventricular Volume,” by H. T. Dodge, H. Sandler, W. A. Baxley, and R. R. Hawley, which was published in The American Journal of Cardiology, Volume 18, July 1966, at pages 10-24. An article entitled “The Architecture of the Heart in Systole and Diastole,” by J. Ross, Jr., E. H. Sonnenblick, J. W. Covell, G. A. Kaiser, and D. Spiro, which was published in Circulation Research, Volume XXI, No. 4, October 1967, at pages 409-412, and an article entitled “Angiocardiographic Determination of Left Ventricular Volume,” by H. Arvidsson, which was published in ACTA Radiologica, Volume 56, November 1961, at pages 321-339, also describe methods for measuring left ventricular volume. A preformed balloon that is just smaller than this chamber size and shape is selected. The balloon device is deflated and allowed to completely collapse as shown in FIG. 4 with the overlapping portions folded over the distal end 24 of the catheter 18. A guide wire is inserted into the femoral artery via a needle, and the needle is withdrawn. A series of increasingly larger cannulas are inserted over the guide wire until a final cannula large enough to admit the folded balloon-catheter tip combination is left in place and the balloon catheter inserted and threaded retrograde, through the Aortic Valve and into the left ventricle. To achieve neutral buoyancy at maximal inflation, an appropriate amount of mercury is introduced via the catheter into the balloon. FIGS. 8 and 9 each illustrate the balloon 16 containing mercury 17. The cannula is then removed. The proximal end 20 of the catheter is connected to the pump 14, which is then activated by an EKG monitoring the patient, so that inflation of the balloon begins with the onset of the left ventricular systole, and is completed at the end of systole. Balloon deflation coincides with the onset of ventricular diastole. In other words, inflation of the balloon occurs during the ventricular systolic interval and deflation occurs during diastole.

The volume of carbon dioxide or helium to be pumped in and exhausted will be determined by assessment of the “dead volume or space” at the end of systole. Various existing techniques, such as ultrasound imaging, or gated isotope scanning may be used to arrive at this volume. The pump will be set so the fully inflated balloon will completely fill the “dead volume”.

This will eliminate the intra-ventricular dead volume created by incomplete systolic contraction of the ventricle. Since Mitral Valve closure and Aortic Valve opening mandate unidirectional flow, this “dead volume” of blood is ejected into the ascending aorta by the kinetic energy of the expanding balloon, and adds to the total ejection volume. It further facilitates diastolic filling of the left ventricle by increasing the negative pressure in the ventricle as the balloon is actively deflated.

The entire sequence described above is repeated with the end of diastole and the beginning of systole.

When used as a permanent “artificial heart”, the balloon is implanted through open heart surgery with the route of entry through the left artrium, so that the catheter traverses the Mitral Valve. As stated previously, it can also be inserted through a small incision in the apex. The catheter is led out through the chest wall and connected to the pump which, of course, is extracorporeal.

FIG. 10 illustrates a modified construction for positioning of the deflated balloon 16 within the catheter 12 during insertion. The largest external diameter during insertion is that of the catheter, while in the construction shown in FIG. 4 the diameter extends to the outer surface 38 of the deflated balloon. The balloon is secured to the inner wall as at 26′. FIGS. 11-13 show the balloon during progressive stages of inflation, and FIG. 14 illustrates the fully inflated balloon.

FIG. 8 illustrates a modified construction in which the entire device, except for the power leads, are implanted subcutaneously. The balloon 16 is connected to a gas reservoir 60 which may be implanted in the abdominal fat and which is surrounded by a solenoid activated electromagnetic bellows-type pump 62. The unit is activated by a control unit 64′ which senses the cardiac electrical cycle. Wires 64, 66 extend through the skin and can be connected to an external power pack (not shown) which may be carried by the patient in a shoulder holster (not shown).

FIG. 9 illustrates another modified construction which is self-contained under the skin of a patient. The balloon 16 is attached to a reservoir 68 positioned within an envelope 70 of skeletal muscle, constructed from either the pectoral or the anterior rectus muscles of the abdomen. This “envelope” or “muscle pump” is paced by a control relay 72 electrically connected by leads 74, 76 to the envelope 70 and the Sinus Node 78 of the heart or the muscle pump may be activated by a standard pacemaker. The relay is powered by a long-life Lithium battery 80. The relay is activated by the Sinus Node and initiates contraction of the muscle pump at the onset of mechanical systole, and relaxation at the onset of diastole.

The balloon 16 used in the construction of FIGS. 8 and 9 is made of thicker material than the reservoirs 60, 68 so that it will normally deflate, thereby inflating the reservoirs.

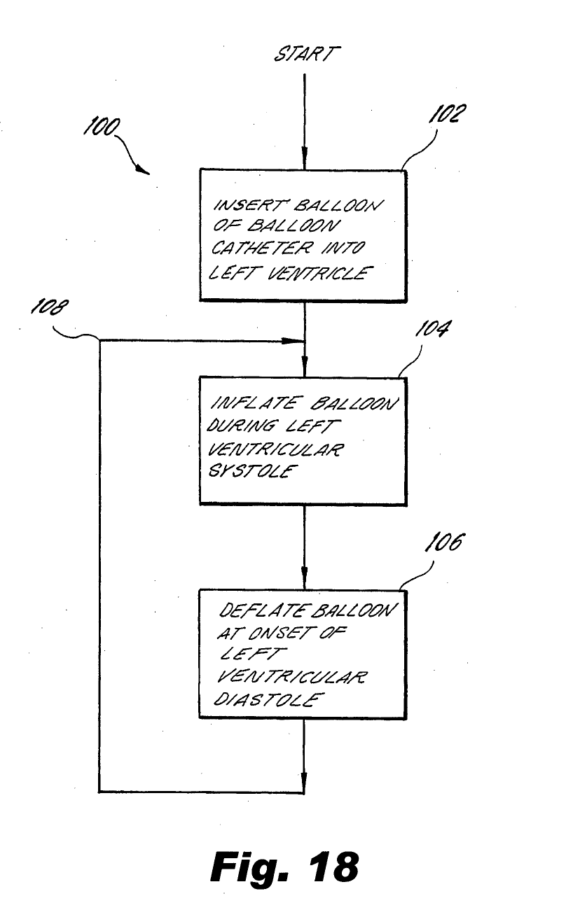

FIG. 18 is a flow chart showing a method of using the heart assist device described herein. The flow chart, which is generally designated by the reference numeral 100, contains a number of blocks. Each block represents a different step of the method. A balloon catheter is inserted into the left ventricle of a patient's heart (block 102). The balloon is inflated during left ventricular systole (block 104), and the balloon is deflated during left ventricular diastole (block 106). Then, the inflating and deflating steps are repeated, as indicated by the line 108.

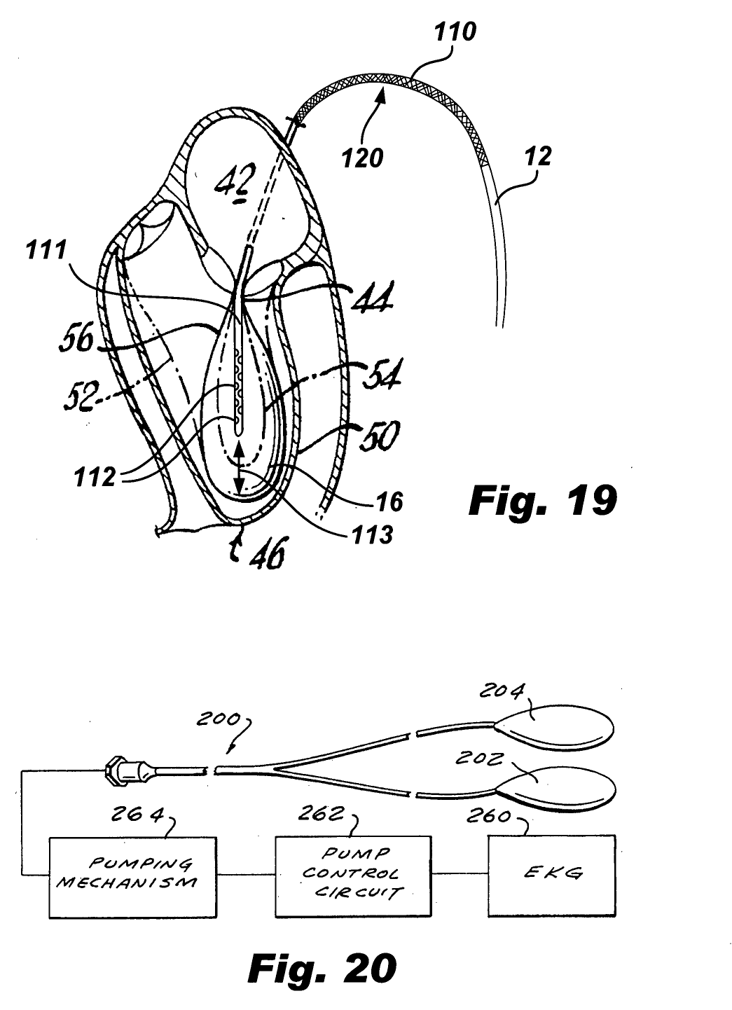

FIG. 19 is a schematic view of a ventricular assist device according to the principles of the present specification. As shown in FIG. 19, the heart assist device is inserted through the left atrium 42 and Mitral Valve 44 into the left ventricle 46. As described above, the balloon 16 expands and contracts to assist the heart, in this example, the left ventricle, to pump blood at or close to full capacity. In FIG. 19, the outer solid line shows the maximum diastolic margin 50 of the inner ventricular wall and the maximum end systolic margin is shown in dotted line 52. The inflated balloon is shown in solid line 56 and the deflated balloon is shown in dotted line 54.

In the example of FIG. 19, the catheter 12 includes a curved portion 120. A wire mesh 110 is provided in the wall of the catheter 12 in at least this curved portion 120 to stiffen the curved portion of the catheter 12. In some examples, the stiffening mesh 110 is included in both the curved portion 120 and the intraventricular portions of the catheter 12. This wire mesh 110 will allow the curved portion 120 of the catheter 12 to be flexibly straightened for insertion through a guide or introducer sheath or cannula, but will cause the curved portion 120 to resume the desired shape and stiffness when released from the introducer sheath or cannula.

Applicants have discovered that stiffening at least the curved portion 120 of the catheter using a stiffening agent, for example, a wire mesh 110 embedded in the wall of the catheter 12, will help keep the balloon 16 in place within the heart and prevent extrusion of the balloon 16 through the aortic valve during systole. Similarly, stiffening the intraventricular portion of the catheter 12 may also further assist to keep the balloon 12 in place and prevent extrusion.

In some examples, the wire mesh 110 is made of a nonmagnetic material, such as titanium or aluminum. Consequently, the patient can be subjected to Magnetic Resonance Imaging (MRI) or other magnetic based diagnostic or therapeutic systems with the heart assist device of FIG. 19 still in place.

Additionally, a tip 111 of the catheter 12 extends into the interior of the balloon 16 in the example of FIG. 19. Holes or openings 1 12 in this catheter tip 1 1 1 allow gas or fluid to be pumped into or out of the balloon 16 from a fluid source 15 (FIG. 1) so as to inflate or deflate the balloon 16. As described above, the inflation and deflation of the balloon 16 is timed by an EKG or other monitoring system to coincide, respectively with systole and diastole.

With the tip 111 of the catheter 12 extending into the balloon 16, it becomes easier to fold the balloon 16 back against the catheter 12 so that the balloon 16 and catheter 12 can be inserted through the lumen of an introducer sheath or cannula and into the heart of the patient. In some embodiments, the introducer sheath or cannula is inserted in a cut down in the femoral artery. The catheter 12 and folded balloon 16 are then moved through the femoral artery to the heart.

Because the present device can be inserted through the femoral artery to the heart, it requires no thoracotomy and can be performed in the Emergency Room or other triage facility to stabilize a patient until that patient can receive a percutaneous transluminal coronary angioplasty or a heart transplant. It is also used to support a “stunned heart” until enzymatic repair occurs to prevent the inevitable death that occurs when the ejection fraction falls below 20%.

Additionally, the catheter tip 111 does not extend to the apex of the balloon. Rather, a separation distance 113 of, for example, 1 cm, separates the end of the catheter tip 111 from the apex of the balloon 16. This prevents damage to the endothelium of that portion of the heart by the catheter tip 111.

In another embodiment, the balloon and catheter of FIG. 19 can be included in a device that includes multiple balloons and catheters such that different areas of the heart can be assisted in their function. For example, an assist balloon may be placed in both the left and the right ventricles of the heart. In another example, an assist balloon may be placed in both the left ventricle and the aorta of the heart, to take advantage of both forward and reverse flow.

As shown in FIG. 20, an EKG 260 monitors the cardiac electrical cycle through a number of leads (not shown) attached to a patient. The EKG 260 sends signals to a pump control circuit 262. The pump control circuit 262 is connected to a pumping mechanism 264. The pump control circuit 262 provides control signals for actuating the pump or pumps in the pumping mechanism 264 so that the balloons 202 and 204 of the heart assist device 200 are inflated and deflated at suitable times during the cardiac electrical cycle, as described above.

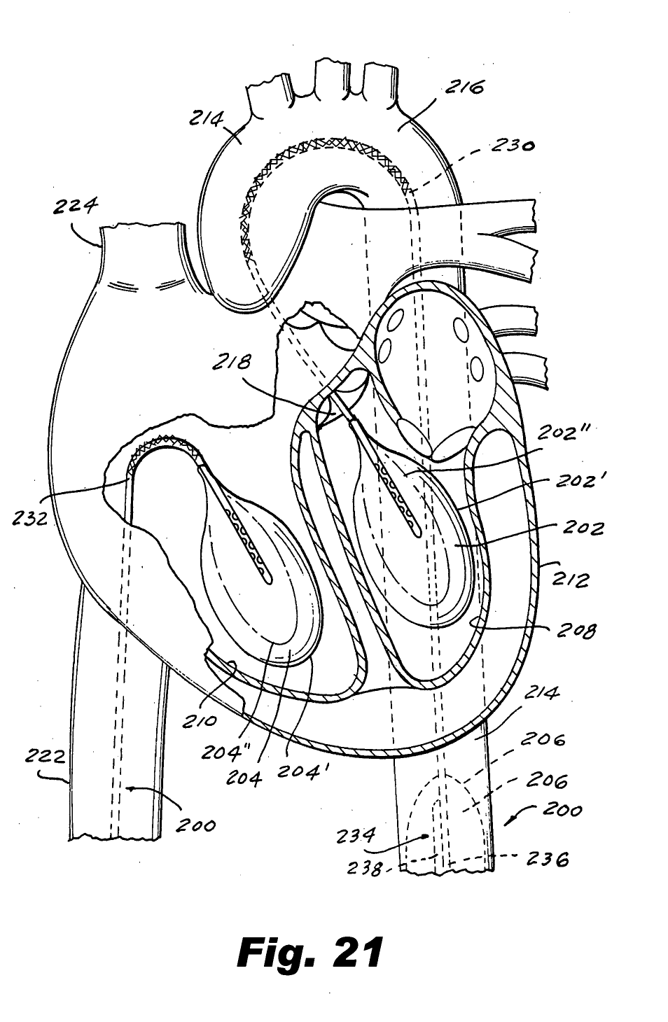

In another example, FIG. 21 shows a heart assist device, which is generally designated by the reference numeral 200, having two intraventricular balloons 202 and 204 and an intraaortic balloon 206. The intraventricular balloons 202 and 204 are inserted into the left ventricle 208 and the right ventricle 210, respectively, of the heart 212. The intraaortic balloon 206 is positioned in the aorta 214 beyond the aortic arch 216. The intraventricular balloon 202 enters the left ventricle 208 through the aortic valve 218, while the intraventricular balloon 204 enters the right ventricle 210 through the tricuspid valve (not shown).

The intraventricular balloons 202 and 204 are connected through tubes 230 and 232, respectively, to a catheter 234, but for ease of illustration, the connection between the tube 232 and the catheter 234 is not shown. The catheter 234 has two lumens 236 and 238, like the catheter 102 discussed previously. The construction of the catheter 234 may be similar to the construction of the catheter 102. The interiors of the intraventricular balloons 202 and 204 communicate through the tubes 230 and 232, respectively, with the lumen 236 of the catheter 234. The interior of the intraaortic balloon 206 communicates with the lumen 238 of the catheter 234. The proximal end of the catheter is connected to a pumping mechanism (not shown), such as the pumping mechanism 116, which is illustrated in FIG. 17 and described above.

The pumping mechanism is controlled to inflate the intraventricular balloons 202 and 204 and deflate the intraaortic balloon 206 during ventricular systole and to inflate the intraaortic balloon 206 during ventricular diastole. The pumping mechanism is controlled to deflate the intraventricular balloons 202 and 204 at about the start of ventricular diastole or at about the end of ventricular systole. The solid lines 202′ and 204′ illustrate the inflated balloons 202 and 204, respectively, while the dashed lines 202″ and 204″ depict the deflated balloons 202 and 204, respectively. The dashed line 206′ shows the inflated balloon 206. The intraventricular balloons 202 and 204 force blood out of the associated ventricle when they are inflating and allow the associated ventricle to fill when they are deflating. The intraaortic balloons 206 urges blood further into the aorta and into the arteries when it is inflating.

As shown in FIG. 21, any or all of the catheters in the device 200 may include a wire mesh or other stiffening agent, particularly through curved portions of the catheters. Additionally, any or all of the catheters may include a tip that extends into the interior of the corresponding balloon with openings in that tip for inflating and deflating the balloon with a fluid or gas. The advantages described above that result from the stiffening agent and the catheter tips that extend into the balloon interiors apply to the devices of FIGS. 20 and 21 as well as to earlier examples.

The preceding description has been presented only to illustrate and describe embodiments and examples of the principles described. This description is not intended to be exhaustive or to limit these principles to any precise form disclosed. Many modifications and variations are possible in light of the above teaching.

What is claimed is:

1. A heart assist device comprising:
a catheter;
a balloon attached to the catheter;
a system for inflating and deflating the balloon in response to systole and diastole of a patient's heart;
wherein said catheter has a curved portion with a stiffening agent disposed therein.

2. The device of claim 1, wherein said stiffening agent comprises a wire mesh embedded in a wall of said catheter.

3. The device of claim 2, wherein said wire mesh is nonmagnetic.

4. The device of claim 3, wherein said wire mesh comprises titanium or aluminum wires.

5. The device of claim 1, further comprising a tip of said catheter that extends into an interior of said balloon.

6. The device of claim 5, wherein said tip of said catheter comprises at least one opening for delivering an inflation gas or fluid through said catheter into said interior of said balloon.

7. The device of claim 5, further comprising a separation between said tip of said catheter and an apex of said balloon when inflated.

8. The device of claim 7, wherein said separation is at least 1 cm.

9. A heart assist device comprising:
a catheter;
a balloon attached to the catheter;
a system for inflating and deflating the balloon in response to systole and diastole of a patient's heart;
wherein said catheter has a tip that extends into an interior of said balloon.

10. The device of claim 9, wherein said catheter comprises a curved portion with a stiffening agent disposed therein.

11. The device of claim 10, wherein said stiffening agent comprises a wire mesh embedded in a wall of said catheter.

12. The device of claim 11, wherein said wire mesh is nonmagnetic.

13. The device of claim 12, wherein said wire mesh comprises titanium or aluminum wires.

14. The device of claim 9, wherein said tip of said catheter comprises at least one opening for delivering an inflation gas or fluid through said catheter into said interior of said balloon.

15. The device of claim 9, further comprising a separation between said tip of said catheter and an apex of said balloon when inflated.

16. The device of claim 15, wherein said separation is at least 1 cm.

17. A method of making a heart assist device comprising:
providing a catheter, wherein said catheter has a curved portion with a stiffening agent disposed therein; and
attaching a balloon to the catheter that can be inflated with a fluid or gas provided through said catheter.

18. The method of claim 17, wherein said stiffening agent comprises a wire mesh embedded in a wall of said catheter.

19. The method of claim 17, further comprising attaching said balloon to said catheter such that a tip of said catheter extends into an interior of said balloon.

20. The method of claim 19, further comprising forming said tip of said catheter with at least one opening for delivering the inflation gas or fluid through said catheter into said interior of said balloon.
